(19) United States
(12) Patent Application Publication
(10) Pub. No.: US 2013/0242096 A1
(43) Pub. Date: Sep. 19, 2013

(54) APPARATUS AND METHOD FOR ENVIRONMENTAL MONITORING

(71) Applicant: Peter David Kingston Mellor, Applecross (AU)

(72) Inventor: Peter David Kingston Mellor, Applecross (AU)

(73) Assignee: Aquadownunder Pty Ltd., Applecross (AU)

(21) Appl. No.: 13/989,109

(22) PCT Filed: Nov. 22, 2011
(86) PCT No.: PCT/AU2011/001500

§ 371 Date: May 23, 2013

(30) Foreign Application Priority Data
Nov. 24, 2010 (AU) 2010905193

(51) Int. Cl. G01N 33/18; H04N 5/225

(52) U.S. Cl. CPC G01N 33/18; H04N 5/2251
USPC 348/143; 348/373

(57) ABSTRACT

An apparatus (10) for environmental monitoring, the apparatus (10) characterised in that it comprises at least one arm (12) mounted in both a rotatable and pivotal manner relative to a base (14), the arm (12) being adjustable in length and having an image capturing device (30) mounted thereon, either directly or indirectly. A method for environmental monitoring is also disclosed.

### TECHNICAL FIELD

The present invention relates to an apparatus and method for environmental monitoring. More particularly, the apparatus and method of the present invention are proposed to have particular application in the monitoring of marine environments, including Benthic Primary Producer (BPP) communities.

### BACKGROUND ART

Presently, environmental regulatory agencies emphasise the preservation of the subsea environment during both the initial environmental impact assessment and the ensuing environmental management phases of project implementation. Projects may be approved on the basis of ‘no effect on adjacent Benthic Primary Producers (BPP) communities’ or its equivalent. Such projects are required to implement reactive management based on BPP condition monitored during implementation. Management triggers have been driven increasingly to the detection of ever decreasing changes in BPP cover or BPP mortality. These changes are sometimes less than just a few percent.

While monitoring techniques are available to detect such changes in BPP, the ability to interpret the results is often limited by inadequate understanding of the ‘normal’ dynamics of these populations. As a consequence, projects may spend a large amount of money establishing extensive and precise monitoring programs that can reveal such small changes in BPP populations that there is no historical basis to interpret them as being either natural or anthropogenic.

Existing methods for the measurement of BPP health include both the Line Intercept Transect (LIT) method and the Multiple Repeated Individual Images (MRII) method.

Line Intersect Method (LIT)—Most marine science research groups use and recommend using sites of replicate 20 m line intersect transects to survey coral reef benthos. With this technique the observer records the length of intersect with the 20 m tape of all benthic organisms beneath it. This method has been recommended and accepted by the Global Coral Reef Monitoring Network (GCRMN), the Intergovernmental Oceanographic Commission (IOC), the United Nations Environment Program (UNEP) and other international agencies. This method is used to obtain an unbiased percentage of live coral, or the relative percent coverage of the benthic life forms, and is described in detail in English et al. (1994). These techniques have been used in many other surveys of benthic communities in the Pilbara and wider Australian region (Worley Parsons Dampier 2009a).

Multiple Repeated Individual Images (MRII)—Coral mortality may be recorded as the death of entire colonies. However, partial mortality, in which a part of the coral colony has died, is more common and is probably more relevant to monitoring impacts on a colonial organism. MRII uses digital still images of entire individual coral colonies (or heads) captured during a series of surveys repeated over time. This process establishes a permanent set of reference coral colonies and records each from as similar an aspect as possible during each survey event.

Images are then scored using Coral Point Count with Excel Extensions (CPCE (Kohler et al. 2006) using a square grid of 64 points. Only points lying within the borders of a coral colony, established from a baseline image, are counted in the scoring. Scoring categories may be varied but routinely include either a measure of live or dead coral. As coral tissue may be obscured by things like sediment, algae or fauna, it is considered safest to score live coral and define ‘partial mortality’ as 1-“live coral”.

These methods all require field measurements and analysis by experienced marine biologists, which is both a time and cost imposition on those conducting the monitoring. Further, there is a health and safety risk where such personnel are required to be in the water.

Remote Operated Vehicles (ROVs) have been utilised previously to undertake studies similar to those noted above. The use of ROVs brings a risk in that low visibility can result in collisions with the BPP. Further, the risk that the same locations, or same coral, are not captured each time the survey is conducted reduces the statistical significance of the results obtained.

It is understood that many of the above problems are shared by other environmental monitoring arrangements and efforts, including other marine environmental situations. The range of specific circumstances in which environmental monitoring is advantageous is understood to be broad.

One object of the method and apparatus of the present invention is to overcome substantially the above mentioned problems of the prior art, or to at least provide a useful alternative thereto.

The preceding discussion of the background art is intended to facilitate an understanding of the present invention only. The discussion is not an acknowledgement or admission that any of the material referred to is or was part of the common general knowledge in any country as at the priority date of the application.

Throughout this specification, unless the context requires otherwise, the word “comprise”, or variations such as “comprises” or “comprising”, will be understood to imply the inclusion of a stated integer or group of integers but not the exclusion of any other integer or group of integers.

### DISCLOSURE OF THE INVENTION

In accordance with the present invention there is provided an apparatus for environmental monitoring, the apparatus characterised in that it comprises a base, an intermediate member, and at least one arm, the intermediate member being mounted by a first end thereof to the base in a rotatable and pivotal manner, the arm being connected to the intermediate member in a pivotal manner at a point remote from the first end, and the arm further being adjustable in length and having an image capturing device mounted directly or indirectly thereon.

Preferably, the mounting of the arm to the base allows rotation of the arm through a full 360 degrees with respect thereto.

Still preferably, the image capturing device is mounted at or near a distal end of the arm relative to the base.

The image capturing device is preferably provided in the form of a camera.

In one form of the present invention the camera is mounted to the arm in a fixed manner.

In a further form of the present invention the camera is mounted on the arm by way of a mounting capable of rotation about two axes, for example by way of a gimbal mount.

Preferably, an intermediate member is provided between the base and the arm. The intermediate member is preferably mounted on the base in a rotatable and pivotal manner. The intermediate member is connected to the arm in a pivotal manner at a point remote from its mounting to the base.

The pivotal movement of the intermediate member relative to the base is preferably driven by way of a first ram provided between the base and the intermediate member, whereby actuation of the first ram controls the angle of the intermediate member relative to the base.

The pivotal movement of the arm relative to the base is preferably achieved by way of a second ram provided between the intermediate member and a point on the arm, whereby actuation of the second ram controls the angle of the arm relative to the intermediate member and in turn the base.

In one form of the present invention the arm is provided as a telescoping arm. In another form of the present invention the arm is provided as an articulated arm.

Preferably, the telescoping or articulation of the arm is achieved through the use or one or more rams provided therein or thereon.

The telescoping or articulation of the arm is, in one form of the present invention, achieved through electric motors.

The image capturing device is preferably received in or on a seat when in a fully contracted position. Preferably, the seat has provided therein or on one or more means to prevent biofouling of the image capturing device. The means to prevent biofouling of the image capturing device may comprise one or more of a UV light source and wipers.

In accordance with the present invention there is further provided a method for environmental monitoring, the method characterised by the reproducible surveying of an environment over a period of time, whereby the surveying is conducted by monitoring and comparing a set of pre-determined points in that environment over that period of time without the need for direct human intervention at the environment during surveying.

Preferably, the method for environmental monitoring is applied to the measurement of Benthic Primary Producer (BPP) health, wherein the environment being monitored is a benthos site and whereby the surveying is conducted by monitoring and comparing a set of pre-determined points at that site over that period of time without the need for direct human intervention at the benthos site during surveying.

Preferably, the method further comprises the conduct of surveys at times that are known to provide appropriate levels of visibility.

Still preferably, the method includes the additional step of conducting either the line intercept transect (LIT) method or the multiple repeated individual images (MRII) method utilising the surveying achieved by the method.

In one form of the present invention the method is conducted through the use of an apparatus for environmental monitoring as described hereinabove.

### BRIEF DESCRIPTION OF THE DRAWINGS

The apparatus for environmental monitoring of the present invention will now be described, by way of example only, with reference to one embodiment thereof and the following drawings, in which:

FIG. 1 is an upper perspective view of an apparatus for environmental monitoring in accordance with the present invention, shown with an arm thereof fully extended, in position in a stylised benthos site;

FIG. 2 is an upper perspective view of the apparatus for environmental monitoring of FIG. 1, shown in a fully retracted position;

FIG. 3 is a side elevational view of the apparatus for environmental monitoring of FIG. 1, shown in a fully retracted position;

FIG. 4 is an upper perspective view of the apparatus for environmental monitoring of FIG. 1, shown in a raised but retracted position;

FIG. 5 is a side elevational view of the apparatus for environmental monitoring of FIG. 1, shown in a raised but retracted position;

FIG. 6 is a side elevational view of the apparatus for environmental monitoring of FIG. 1, shown in a raised and fully extended position;

FIG. 7 is a side elevational view of the apparatus for environmental monitoring of FIG. 1, shown in a raised, extended position, demonstrating how the apparatus may be used to provide access to a point in the environment that might otherwise not be accessible.

### BEST MODE FOR CARRYING OUT THE INVENTION

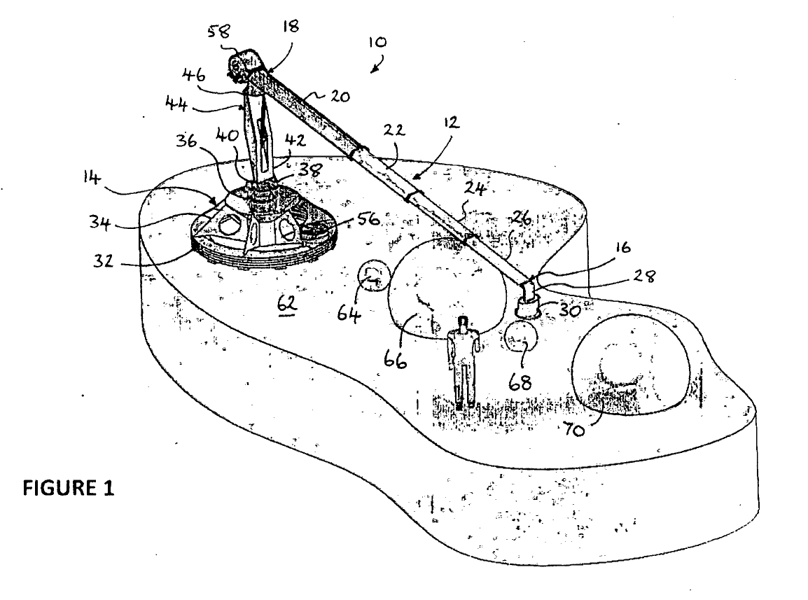

In FIGS. 1 to 7 there is shown both an apparatus 10 and a method for environmental monitoring, apparatus comprising an arm 12 mounted relative to a base 14. The arm 12 has a distal end 16 remote from the base 14, and a proximal end 18 adjacent the base 14. The arm 12 comprises four (4) telescoping portions, each being received one within the other in known manner, the portions being a proximal portion 20, a first intermediate portion 22, a second intermediate portion 24 and a distal portion 26.

The distal end 16 of the arm 12 has provided thereon a fixed mount 28 for an image capturing device, for example an xyz camera 30.

The base 14 comprises a lowermost portion 32, provided in annular form, and an intermediate portion 34 arranged to engage the lowermost portion 32 and on which is provided a rotatable mounting 36. The rotatable mounting 36 supports, in a rotatable manner relative to the remainder of the base 14, a platform 38.

On the platform 38 is provided a pivotal mounting 40 for a first end 42 of an intermediate member 44. At a second end 46 of the intermediate member 44 there is provided a pivotal mounting 48 for the proximal end 18 of the arm 12.

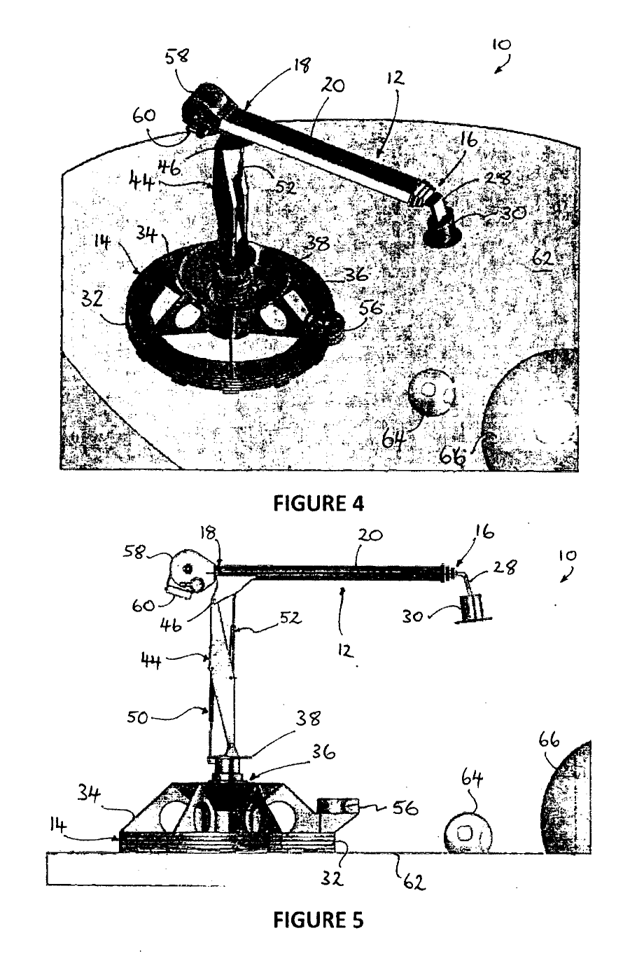

A ram 50 is provided extending between a point on the platform 38 of the base 14 and a intermediate point on the intermediate member 44, such that actuation of the ram 50 causes the intermediate member 44 to pivot relative to the base 14, in turn raising the second end 46 relative to the base 14, further thereby raising at least the proximal end 18 of the arm 12, as best seen in FIGS. 4 and 5. The Applicant envisages that this action may raise the proximal end 18 of the arm 12 to a height of at least 2 metres.

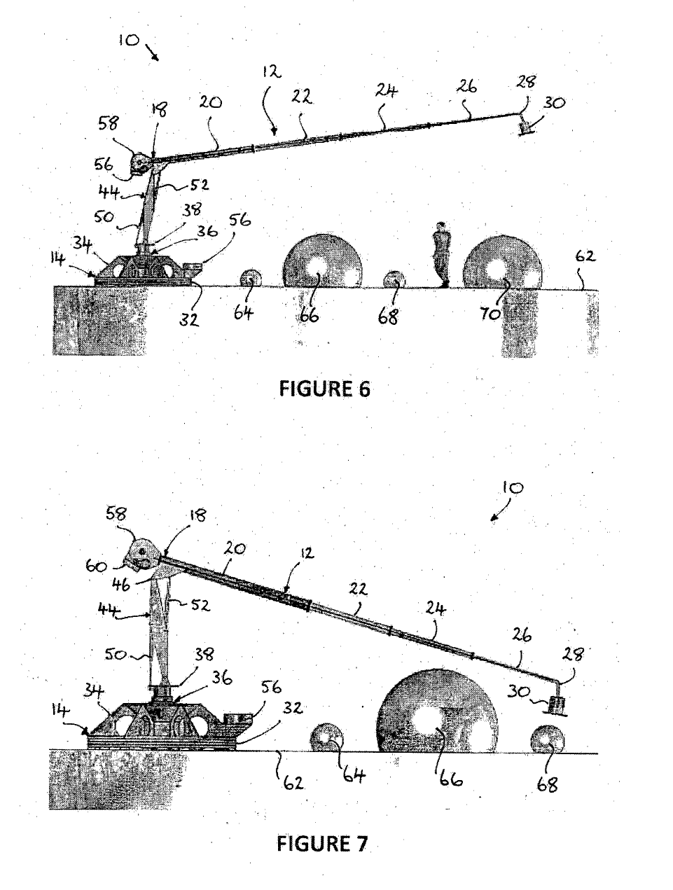

A ram 52 is provided extending between an intermediate point on the intermediate member 44 and a mounting point 54 provided under the proximal end 18 of the arm 12, best seen in FIG. 5. Actuation of the ram 52 causes the arm 12 to pivot relative to the intermediate member 44 and in turn relative to the base 14. This has the effect of causing the arm 12 to pivot upwardly and downwardly, as may be seen referring to FIGS. 6 and 7, for example.

It is envisaged that the rams 50 and 52 may be either hydraulic or pneumatic in operation.

Further rams (not shown) may be provided to power the extension or retraction of the portions 20, 22, 24 and 26 of the arm 12. Alternatively, the extension and retraction of the portions 20, 22, 24 and 26 of the arm 12 may be powered by way of electrics, with the necessary cabling and motors (not shown) being provided within the portions 20, 22, 24 and 26.

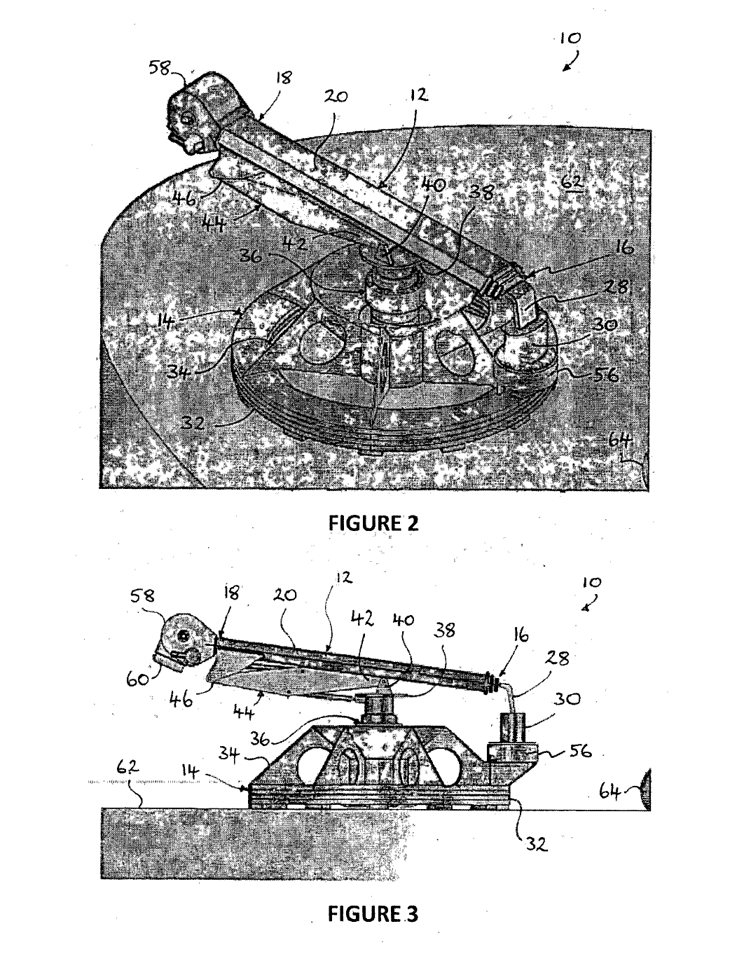

A seat 56 is provided on the base 14 for the image capturing device. When the apparatus 10 is in a fully contracted position, as seen in FIGS. 2 and 3, the camera 30 is received of docked in the seat 56, by which fouling of the camera by the surrounding environment may be minimised. Further, the camera seat 56 has provided therein means to prevent biofouling of a lens of the camera 30. The means to prevent biofouling may comprise a UV light source and wipers, whereby when the camera 30 is docked in the seat 56 the UV light source and wipers act on the lens of the camera to keep it free of marine growth.

A housing 58 is provided at the proximal end 18 of the arm 12. The housing 58 has provided thereon an access hatch 60, best seen in FIGS. 3 and 5, by which maintenance on the items housed therein may be achieved. The drive mechanism for the portions of the arm 12 is in part housed within the housing 58.

In use, a remote operator or program is able to control the movement of the rotatable mounting 36, the intermediate member 44, and the arm 12, and finally in turn the position of the camera 30 relative to a sub-sea surface 62 and benthos features 64, 66, 68 and 70, depicted in stylised form, such as may be present, best seen in FIG. 1, so that a desired series of points/positions of BPP are monitored consistently over time. That is, a user determined transecting of the BPP can be conducted, regularly if desired, in a reproducible manner. This reproducibility arises from the fact that the operation is governed entirely by coordinates/positions programmed by the user and which is then delivered or carried out by the apparatus 10 in response to same in a mechanical fashion. This reproducibility further ensures substantially the reliability of the survey results obtained. The significance of resulting statistical data is improved relative to methods of the prior art.

The camera 30 may be provided as either a stills or video camera, dependent upon the nature of the analysis that is desired by the users. Similarly, the images captured by the camera may be analysed either live or at a later time, again as desired by the users.

As noted above, the apparatus of the present invention will be coupled with a specific mission, for example 22 degrees 6 meters etc., to enable precision capture of the same BPP repeatedly.

As can be seen from FIGS. 6 and 7, the operation of the intermediate member 44 and arm 12 is sufficiently flexible to allow accurate and reproducible interaction with, for example, benthos feature 68, despite the intervening larger benthos feature 66.

It is envisaged that the arm 12 may comprise more or less telescoping portions than described hereinabove without departing from the scope of the present invention.

It is further envisaged that the arm 12 may be provided in the form of an articulated arm whereby the arm folds on itself, in addition to the provision of the ‘folding’ intermediate member 44, without departing from the scope of the present invention.

It is yet still further envisaged that a gimbal mount for the camera 30 may be provided in place of the fixed mount 28 described above. Such a gimbal mount would be such that it provided for rotation of the camera 30 around two axes, being the axis described generally by the orientation of the arm 12, and that described by a pair of arms that held the camera 30 therebetween, like pincers arranged in a plane horizontal to that described by the arm 12, in a rotatable manner. However, the fixed mount 28 described above is preferred by the Applicants due to the simplicity thereof, which in a generally corrosive marine environment can be a distinct advantage.

In can be seen from the forgoing that the apparatus for environmental monitoring of the present invention provides a basis on which the traditional monitoring techniques can be carried out in a reproducible and regular manner without many of the problems associated with prior art methods of enacting those techniques.

Modifications and variations such as would be apparent to the skilled addressee are considered to fall within the scope of the present invention. For example, it is envisaged that the apparatus and method of the present invention may have application in the monitoring of well heads in an underwater or marine environment. It is envisaged that other circumstances are similarly suited to application of the apparatus and method of the present invention.

### References

English. S, Wilkinson. C, et al. (1994). Survey Manual for Tropical Marine Resources. Townsville, Australia.
Kohler, K. E. and S. M. Gill (2006). “Coral Point Count with Excel extensions (CPCe): A visual basic program for the determination of coral and substrate coverage using random point count methodology.” Computers and Geosciences 32: 1259-1269.
WorleyParsons (2009a). Comparison of the Dampier Port Fringing Reef Benthic Community with Nearby Reef Areas. Submitted as an appendix of the DMSF ARI.

1. An apparatus for environmental monitoring, the apparatus characterised in that it comprises a base, an intermediate member, and at least one arm, the intermediate member being mounted by a first end thereof to the base in a rotatable and pivotal manner, the arm being connected to the intermediate member in a pivotal manner at a point remote from the first end, and the arm further being adjustable in length and having an image capturing device mounted directly or indirectly thereon.

2. An apparatus according to claim 1, wherein the mounting of the arm to the base allows rotation of the arm through a full 360 degrees with respect thereto.

3. An apparatus according to claim 1, wherein the image capturing device is mounted at or near a distal end of the arm relative to the base.

4. An apparatus according to any one of claims 1, wherein the image capturing device is provided in the form of a camera.

5. An apparatus according to claim 4, wherein the camera is mounted to the arm in a fixed manner.

6. An apparatus according to claim 4, wherein the camera is mounted on the arm by way of a mounting capable of rotation about two axes.

7. An apparatus according to claim 6, wherein the camera is mounted on the arm by way of a gimbal mount.

8. (canceled)

9. (canceled)

10. (canceled)

11. An apparatus according to claim 1, wherein the pivotal movement of the intermediate member relative to the base is driven by way of a first ram provided between the base and the intermediate member, whereby actuation of the first ram controls the angle of the intermediate member relative to the base.

12. An apparatus according to claim 1, wherein the pivotal movement of the arm relative to the base is achieved by way of a second ram provided between the intermediate member and a point on the arm, whereby actuation of the second ram controls the angle of the arm relative to the intermediate member and in turn the base.

13. An apparatus according to claim 1, wherein the arm is provided as a telescoping arm.

14. An apparatus according to claim 1, wherein the arm is provided as an articulated arm.

15. An apparatus according to claim 1, wherein the arm is a telescoping or articulated arm, and wherein the telescoping or articulation of the arm is achieved through the use or one or more rams provided therein or thereon.

16. An apparatus according to claim 1, wherein the arm is a telescoping or articulated arm, and wherein the telescoping or articulation of the arm is achieved through electric motors.

17. An apparatus according to claim 1, wherein the image capturing device is received in or on a seat when in a fully contracted position.

18. An apparatus according to claim 17, wherein the seat has provided therein or on one or more means to prevent biofouling of the image capturing device.

19. An apparatus according to claim 18, wherein the means to prevent biofouling of the image capturing device comprises one or more of a UV light source and wipers.

20. A method for environmental monitoring, the method characterised by the reproducible surveying of an environment over a period of time, whereby the surveying is conducted by monitoring and comparing a set of pre-determined points in that environment over that period of time without the need for direct human intervention at the environment during surveying.

21. A method according to claim 20, wherein the method for environmental monitoring is applied to the measurement of Benthic Primary Producer (BPP) health, wherein the environment being monitored is a benthos site and whereby the surveying is conducted by monitoring and comparing a set of pre-determined points at that site over that period of time without the need for direct human intervention at the benthos site during surveying.

22. A method according to claim 20, wherein the method further comprises the conduct of surveys at times that are known to provide appropriate levels of visibility.

23. A method according to claim 20, wherein the method includes the additional step of conducting either the line intercept transect (LIT) method or the multiple repeated individual images (MRII) method utilising the surveying achieved by the method.

24. (canceled)

25. (canceled)

26. (canceled)
